(19) United States
(12) Patent Application Publication
(10) Pub. No.: US 2018/0161338 A1
(43) Pub. Date: Jun. 14, 2018

(54) RETOSIBAN FOR THE TREATMENT OF PRE-TERM LABOUR

(71) Applicant: Gordon Campbell Sinclair SMITH, Cambridge (GB)

(72) Inventor: Gordon Campbell Sinclair SMITH, Cambridge (GB)

(21) Appl. No.: 15/892,057

(22) Filed: Feb. 8, 2018

Related U.S. Application Data
(63) Continuation of application No. 15/319,575, filed on Dec. 16, 2016.

(51) Int. Cl. A61K 31/5377; A61K 9/00

(52) U.S. Cl. CPC A61K 31/5377; A61K 9/0053; A61K 9/0019

(57) ABSTRACT

The present invention relates to a method of treating pre-term labour with retosiban in subjects with conditions resulting in uterine overdistension including polyhydroamnios and multiple gestation. In another aspect, the invention relates to a method of preventing pre-term labour in subjects by the prophylactic administration of retosiban.

### CROSS-REFERENCE TO RELATED APPLICATIONS

This application is a continuation of U.S. application Ser. No. 15/319,575 filed Dec. 16, 2016, which is a National Phase Application of International Application No. PCT/EP2014/062602 filed Jun. 16 2014.

### FIELD OF THE INVENTION

The present invention relates to a method of treating pre-term labour with retosiban in subjects with conditions resulting in uterine overdistension including polyhydroamnios and multiple gestation. In another aspect, the invention relates to a method of preventing pre-term labour in subjects by the prophylactic administration of retosiban.

### BACKGROUND OF THE INVENTION

The process of human parturition or birth involves a number of distinct physiological changes. This includes strong co-ordinated contraction of the myometrium and dilation of the cervix. In addition, the chorioamniotic membranes surrounding the foetus must separate from the decidua (the lining of the uterus) to permit post partum expulsion of the membranes. Unlike certain other mammals where labour is triggered by withdrawal of progesterone, in humans there would appear to be no single trigger of all these events. Instead it has been postulated that a “modular accumulation of physiological systems” must occur for labour and delivery to occur, namely (1) activation of the myometrium such that it is capable of responding to contractile stimuli in a co-ordinated manner, (2) a change in the mechanical properties of the cervix such that it is capable of being dilated following uterine contraction (this is typically termed “cervical ripening”) and (3) biochemical changes to the chorioamniotic membranes (Mitchell and Taggart, Am J Physiol Regul Integr Comp Physiol, 2009, 297: R525-R545).

Once all of these systems are in place, contractions of the smooth muscle cells of the myometrium will be capable of dilating the uterus and expelling the foetus and foetal tissues (placenta/membranes). The mechanism of smooth muscle contraction has been extensively studied and involves filaments of myosin moving relative to filaments of actin (this is termed the “sliding filament model”) that ultimately results in a shortening (or contraction) of the cell. The relative movement is accomplished by conformation changes that accompany phosphorylation (and ADP binding) and dephosphorylation (and ATP binding) of the myosin regulatory light chain. Regulation of muscle contraction is achieved by regulation of the enzymes that phosphorylate (myosin light chain kinase or MLCK) and dephosphorylate (myosin light chain phosphatase or MLCP) the myosin regulatory light chain. Regulation of MLCK is achieved by regulation of the level of intracellular Ca²⁺. A notable property of the smooth muscle cells in the myometrium is that they have unstable membrane potentials that periodically result in influx of extracellular Ca²⁺ through ion channels. This results in elevated intracellular Ca²⁺ levels that are required for contraction (Mitchell and Taggart, supra).

It will also be appreciated that intracellular Ca²⁺ is a second messenger in a very large number of signalling pathways. Pathways that elevate levels of intracellular Ca²⁺ (either through receptor operated Ca²⁺ entry through the plasma membrane or through Ca²⁺ release from the sarcoplasmic reticulum) may thus lead to augmentation of contraction. A number of G protein coupled receptors have been postulated to be involved in augmenting intracellular Ca²⁺ levels during labour including the receptors for OT (oxytocin - note that synthetic oxytocin is frequently used during labour to increase the frequency and intensity of contractions), ET-1 (endothelin-1) and certain prostaglandins including PGF-2α and PGFH2 (prostaglandin F2α and prostaglandin H2; Mitchell and Taggart, supra). It is entirely conceivable that numerous other signalling pathways may be involved in augmenting intracellular Ca²⁺ levels in uterine smooth muscle cells during labour in a similar manner.

Signalling pathways that result in inhibition of MLCP leads to a prolongation of the phosphorylated form of the myosin regulatory light chain (the contractile form) and hence result in enhanced myometrial contractility. There is some evidence to suggest that pathways that elevate levels of the second messenger diacylglycerol (DAG) indirectly inhibit MLCP (Mitchell and Taggart, supra). In addition, there is some evidence to suggest that signalling pathways that elevate levels of ROK (rho associated kinase) result in inhibition of MLCP and hence enhanced myometrial contractility (Mitchell and Taggart, supra).

In addition to regulatory pathways resulting in myometrial contractility, relaxation pathways also exist. Phosphorylation of MLCK inhibits phosphorylation of the myosin regulatory light chain resulting in uterine relaxation. Signalling pathways that elevate intracellular cAMP or cGMP result in phosphorylation of MLCK and hence uterine relaxation. These include the β₂-adrenergic pathway, the PGI2 (prostaglandin I2, also known as prostacyclin) pathway and the nitric oxide (NO) pathway (Mitchell and Taggart, supra).

It will be apparent that, for such signalling pathways to be effective in regulating myometrial contractility, the necessary ligands, receptors and machinery capable of transducing the signals generated on ligand binding must be present in the smooth muscle cells. Several studies have shown that the complement of signalling molecules in the myometrium changes as pregnancy progresses, although there is frequent disagreement on the nature of the changes of particular proteins (Mitchell and Taggart, supra). For example, some studies show that concentrations of the oxytocin receptor in the myometrium increase before labour onset, but this is not found in other studies. Microarray techniques have been used to assess changes in gene expression at parturition. These studies have shown significant changes in the expression of genes associated with the immune system and in a variety of signalling pathways (Mitchell and Taggart, supra). It thus seems plausible that the balance between contractile and relaxation signalling pathways is, at least in part, regulated by the protein content of the cell. In other words, there may be a shift in the protein content of the myometrium to favour relaxation pathways during early gestation, but to favour contractile pathways at term. How these changes in levels of gene expression are regulated is unknown, but it is noted that progesterone is believed to increase expression of genes associated with uterine relaxation and suppress genes associated with uterine activation and some investigators have proposed that there is a “functional” progesterone withdrawal prior to the initiation of labour in which the concentration of progesterone in the myometrium is reduced despite elevated levels in maternal plasma (several mechanisms have been proposed that would be capable achieving this, although there is little evidence supporting any particular mechanism at the present time, see Mitchell and Taggart, supra).

For a successful delivery, it is not sufficient that individual smooth muscle cells have the appropriate transduction machinery to enable it to respond to contractile signals. It is also necessary that the myometrium as a whole is capable of co-ordinated contraction. This would appear to be regulated at least in part by prostaglandins, the levels of which increase prior to the onset of labour or during early labour. Prostaglandins stimulate myometrial gap junction formation that enables rapid and efficient spread of electrical impulses throughout the uterine muscle. It will be appreciated that, in addition to stimulating myometrial gap junction formation, prostaglandins are also capable of augmenting intracellular Ca²⁺ levels, promoting myometrial contraction. This may help to co-ordinate the various changes required for myometrial activation.

The second physiological change that must occur for successful delivery is cervical ripening. During most of pregnancy, the cervix is a rigid organ that helps to support the weight of the growing foetus. Its rigidity is due to its high content of collagen and other structural molecules (e.g. proteoglycans), as well as to the selection of rigid forms of these structural molecules (e.g. the proteoaminoglycan decorin—PGS2—which covers the surface of collagen bundles stabilising them and promoting the formation of thicker bundles of fibres; Romero et al., Ann N Y Acad Sci, 1994, 734:414-429). Cervical ripening appears to result from a decrease in total collagen content, a change in the nature of the collagen and other structural molecules present (e.g. PGS2 is replaced with PGS1 that has no affinity for collagen, resulting in disorganised collagen fibrils with little rigidity) and an increase in collagenolytic activity (Romero et al., supra). Cervical ripening is known to be accompanied by an influx of pro-inflammatory cells, particularly neutrophils into the cervix. It is believed that these cells secrete matrix metalloproteinases that breakdown the collagen matrix as well as cytokines and other mediators such as prostaglandins that have an effect on extracellular matrix metabolism (Romero et al., supra). In this regard, it is noted that prostaglandins are used clinically to induce cervical ripening prior to induction of labour or abortion (Romero et al., supra). In addition, there is evidence for a potential role for estrogen (IV administration of 17β estradiol induces cervical ripening and estrogen is known to stimulate collagen degradation in vitro; Romero et al., supra) and anti-progestins are also used clinically for cervical ripening (progesterone has been shown to block estrogen induces collagenolysis in vitro; Romero et al., supra). As mentioned above, it has been proposed that there is a functional progesterone withdrawal prior to the initiation of labour, and if this occurs, this would remove a signalling pathway that has a negative effect on cervical ripening and may provide some mechanism to co-ordinate myometrial activation and cervical ripening.

The final physiological change required for successful delivery is the separation of the chorioamniotic membrane from the decidua. Whilst it is known that this is achieved by dissolving the fibronectin cement linking the maternal and foetal tissues, little is known about the regulation of this process (Romero et al., supra).

It follows from the above that labour will take place once the myometrium is activated to respond to contractile stimuli in a co-ordinated manner, and once the changes resulting in cervical ripening and separation of foetal and maternal membranes have been initiated. Although there are a few instances (highlighted above) where the same signals are involved in activating the separate physiological systems required for labour, it is not clear how much overlap or coordination exists between the mechanisms governing each physiological system. Of course, the motivation for understanding the regulation of parturition relates to the serious clinical consequences associated with being born too early. Pre-term births (deliveries that occur at less than 37 weeks gestation) account for 75% of neonatal mortality and more than half the long term morbidity. Although most pre-term babies survive, they are at increased risk of neurodevelopmental impairments and respiratory and gastrointestinal complications (Goldenberg et al., Lancet, 2008, 371: 75-84).

Although most instances of pre-term labour appear to be spontaneous, pre-term labour is associated with a number of risk factors (several of which may be present in a single subject). These include intra-uterine infection or inflammation, precocious foetal endocrine activation, short cervix, maternal history of pre-term delivery, decidual haemorrhage, excessive myometrial and foetal membrane overdistension (caused by multiple gestation or polyhydroamnios—note that stretch of the uterus is hypothesised to have a role in labour induction at term) and stress (Goldenberg et al., supra). Intra-uterine infection triggers a number of signaling pathways. For example, microorganisms are recognized by receptors that elicit release of cytokines which in turn stimulate the production of prostaglandins, other inflammatory mediators and matrix degrading enzymes (Goldenberg et al., supra). Microbial endotoxins are also known to stimulate the production of prostaglandins (Goldenberg et al., supra). As outlined above, prostaglandins are known to be involved in myometrial contraction and cervical ripening. Inflammation is also associated with elevated levels of inflammatory cytokines, IL-1β (interleukin 1β), IL6 (interleukin 6) and TNFα (tumor necrosis factor a) which are believed to initiate signalling pathways that activate the necessary physiological systems for labour. The link between uterine stretch and labour induction will be discussed later. The signalling pathways linking the remaining risk factors and the required physiological systems is not clear.

Since the majority of cases of pre-term labour are spontaneous and because it is not known how most of the risk factors for pre-term labour activate the physiological systems required for labour, therapeutic interventions for pre-term labour rely on inhibiting uterine contractions or promoting uterine relaxation by modulating the signalling pathways that regulate the activity of MLCK and MLCP (see above; Simhan and Caritis, N Engl J Med, 2007, 357(5): 477-487). Drugs that inhibit uterine contractions/promote uterine relaxation are known as tocolytics. Drugs currently used as tocolytics have a responder rate of less than 50%. Further, the responder rate to tocolytics is somewhat misleadingly high since it includes cases of “false” labour - which resolves spontaneously without intervention—which is difficult to distinguish from pre-term labour based on clinical impression alone. It follows that, in the majority of cases, tocolytic therapy is not capable of stopping uterine contractions or interrupting the labour process. This is probably because, in these patients, the preponderance of active signalling pathways regulating MLCK and MLCP promote contraction.

Those patients that respond to the drugs used as tocolytics typically have their pregnancies maintained for a relatively short period (24-48 hours; Simhan and Caritis, supra), although the Cochrane review for nifedipine indicates that it significantly delays delivery when assessed at 7 days. The short duration of action of most tocolytics suggests that there is no change to the underlying status of the myometrium such that labour (re-)initiates following withdrawal of therapy. Nonetheless, this short period tocolysis is considered important since it permits the patient to be moved to a tertiary care centre which can provide the best care for the mother and baby (although there are no studies supporting this hypothesis). It also may allow time for the administration and therapeutic effect of corticosteroids.

Retosiban ((3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione) is a compound capable of binding to the oxytocin receptor (WO2005/000840). It is in clinical development for the acute treatment of pre-term labour in singleton pregnancies. In a phase II clinical study, retosiban had a responder rate slightly higher than other tocolytics (the response rate or percentage of women achieving uterine quiescence (≤4 contractions/h with no change in cervical dilation>1 cm at 5-6 h) was 50-57%, depending upon route of administration/dose; see http://www.gsk-clinicalstudyregister.com/study/OTA105256#rs). In this same study, it was shown that retosiban increased days to delivery by a mean of 8.2 days relative to placebo (Snidow et al., Am J Obstet Gynecol., 2013, 2: S155).

True responders in this study (i.e. responders in the study excluding those in false labour—see above) presumably represent those subjects where the contractile stimulus promoting labour was primarily mediated by the oxytocin pathway (such that inhibiting this pathway puts the myometrium into a state where relaxation pathways are favoured). This is evidence that the oxytocin pathway is activated in some women in pre-term labour. However, what triggers this pathway in these women is usually unclear.

As mentioned above, pre-term labour is associated with uterine overdistension caused by multiple gestation or polyhydroamnios. Since it is also believed that stretch may play a role in the normal initiation of labour at term, it is possible that this simply reflects early induction of this pathway in subjects having uterine over-distension.

Stretching myometrial strips has been demonstrated to increase their contractile response to KC (which causes contraction by depolarization of the uterine smooth muscle cells, and by opening of voltage sensitive channels leading to an increase in intracellular Ca²⁺) and oxytocin (which increases intracellular Ca²⁺ levels as discussed above and also activates plasma membrane Ca²⁺ channels; Tattersall et al., Reprod Sci, 2010, 17(3 Suppl): 85A). This suggests that stretch may play a role in myometrial activation (one of the physiological changes required for the initiation of labour). Microarray techniques have been used to identify transcripts whose levels in myometrial strips are significantly different when incubated in high and low stretch conditions. A large number of rather diverse transcripts (e.g. gastrin releasing peptide) were identified (Tattersall et al., J Physiol., 2012, 590(9): 2081-2093). The pathway by which these gene products are regulated is not known, nor indeed is it known if there is a single or several signalling pathways involved in regulating of all the gene products identified. One noteable gene product that did not appear to be differentially regulated is the oxytocin receptor. A large number of studies show that levels of the oxytocin receptor are increased during labour, although there are conflicting findings about the precise timing of this increase with some studies suggesting that the increase occurs in late gestation and other studies suggesting that this occurs at the onset of labour (Terzidou et al., J Clin Endocrin Metab, 2005, 90(1): 237-246). If the former explanation is true, then it is possible that the non-identification of the oxytocin receptor as being regulated by stretch could simply be attributed to the fact that it was already elevated in the tissues concerned (which were obtained from pregnant woman undergoing elective caesarean section at term, where levels of the oxytocin receptor were initially high). Of course, if this is the case, it seems clear that the increase in oxytocin receptor, whilst possibly triggered by stretch (there is some evidence that stretch activated focal adhesion proteins might be involved in activation of MAPK signalling cascades which result in an increase in the binding of C-EBP to sites in the oxytocin receptor promoter; Li et al., PLoS ONE, 2009, 4(1): e7489), cannot alone be responsible for labour induction (since high levels are present before labour is initiated). It may simply represent one change involved in myometrial activation (the biochemical changes that take place to permit the myometrium to respond to contractile stimuli). Even supposing that the upregulation of the oxytocin receptor was directly in the pathway transducing the initiation of labour, it would seem logical that its ligand, oxytocin, would also need to be present. There is no evidence suggesting that oxytocin levels are regulated by stretch.

There are isolated reports of atosiban (an oxytocin antagonist) being used successfully to treat pre-term labour in twin pregnancies. However, to the best of our knowledge, no placebo controlled clinical trials have been conducted to investigate the effect of oxytocin ligands in subjects with multiple gestations or polyhydroamnios (in retosiban clinical trials conducted to date, such subjects were excluded).

One study compared the contractile properties of myometrium obtained from the uterus of women with singleton and twin pregnancies during elective caesarian section (Turton et al., PLoS ONE; 8 (5): e63800). This study showed that myometrial contractile activity in response to oxytocin was shown to correlate with neonatal birth weight (the birth weight of twins was combined), a marker of uterine stretch. Whilst this would appear to suggest a role for the oxytocin pathway in pre-term labour associated with uterine overdistension, the very same study shows no significant differences in contractile activity in response to oxytocin in myometrium derived from a pre-term twin pregnancy compared to myometrium derived from a pre-term singleton pregnancy.

Accordingly, it is not clear whether inhibition of the oxytocin pathway would be effective in treating pre-term labour in women with uterine overdistension.

In this regard, it is noted that the fact that oxytocin antagonists are effective in treating pre-term labour in a proportion of females with singleton pregnancies cannot be used to predict efficacy in treating pre-term labour in subjects with a multiple gestation. This is because it is known that therapies effective in preventing pre-term labour in singleton pregnancies are not always effective in preventing pre-term labour in multiple gestations. For example, in clinical studies addressing the role of progesterone supplementation in preventing pre-term delivery it was found that daily administration of vaginal progesterone reduced by more than 40% the frequency of birth before 34 weeks of gestation among asymptomatic women with a short cervix (a risk factor for pre-term labour) that were pregnant with a single foetus (Fonseca et al., N Engl J Med, 2007, 357: 462-469). By contrast, other studies shows no reduction in the frequency of pre-term birth following prophylactic administration of intramuscular 17-α hydroxyprogesterone caproate in women pregnant with twins (Rouse et al., N Engl J Med, 2007, 357: 454-461) or following prophylactic administration of vaginal progesterone gel in women pregnant with twins (Norman et al., Lancet, 2009, 373(9680): 2034-2040). Further, cerclage (placing a stitch in the cervix) has been shown to be beneficial in singleton pregnancies where the mother has a short cervix and prior pre-term birth (both risk factors for pre-term labour) but not to be beneficial in multiple pregnancies where the mother has a short cervix (Berghella et al., Obstet Gynecol, 2005, 106(1): 181-189).

These differences in response to prophylactic therapies aimed at preventing pre-term labour suggest that the mechanisms by which the physiological systems required for labour may be activated differently in women with multiple gestations compared to at least the majority of women with singleton pregnancies.

FIGS. 1-3 of the present patent application were disclosed by Dr Alex Moraitis in a poster (Moraitis et al., Retosiban prevents stretch-induced stimulation of human myometrial contractility) at a closed meeting of the Blair Bell Society at the Royal College of Obstetricians and Gynaecologists that took place between Monday 16^{th} to Tuesday 17 Dec. 2013. Dr. Moraitis conducted the experiments disclosed in the Example under the direction of the inventor, Professor Gordon Smith.

### SUMMARY OF THE INVENTION

The present invention provides, in a first aspect, a method of treating a human female subject with a multiple gestation or polyhydroamnios in pre-term labour, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject.

In a second aspect, the invention provides a method for preventing pre-term labour in a human female subject with at least one recognized risk factor for pre-term labour, which method comprises administering (3R,6R)-3-(2,3-dihydro-1H-inden-2-yl)-1-[(1R)-1-(2-methyl-1,3 -oxazol-4-yl)-2-(morpholin-4-yl}-2-oxoethyl]-6-[(1S)-1-methylpropyl]-2,5-piperazinedione to the human female subject.

### BRIEF DESCRIPTION OF THE DRAWINGS

FIG. 1 shows the effect of stretch on maximal contractile responses to KCl and oxytocin in human pregnant myometrium incubated with retosiban or vehicle.

FIG. 2 shows the effect of retosiban on maximal contractile responses to KCl and oxytocin in human pregnant myometrium incubated under low or high stretch.

FIG. 3 shows the correlation between the effect of stretch and the effect of retosiban. A=KCl. B=oxytocin.

FIG. 4 shows the effect of retosiban on the pEC₅₀ to oxytocin. A=low stretch. B=high stretch.

### DETAILED DESCRIPTION OF THE INVENTION

The Example describes an experiment in which myometrial explants are maintained in a viable state for a period of up to 3 days under conditions of high and low stretch in the presence or absence of retosiban. Following removal of retosiban, the myometrial explants were challenged with KCl (which depolarizes the cell membrane resulting in smooth muscle contraction) and oxytocin (which triggers contraction of smooth muscle by triggering the release of intracellular Ca²⁺—see background).

The key finding is that in conditions of high stretch (simulating uterine overdistension), retosiban inhibited myometrial contractility resulting from KCl or oxytocin challenge. This implies that the preincubation with retosiban in some way changed the signalling pathways of the myometrium in such a way as to prevent it responding to contractile stimuli. It is noteworthy that these results cannot result simply from retosiban blocking the oxytocin receptor (this is for two reasons: (1) retosiban was removed before KCl and oxytocin challenge and (2) KCl does not exert its contractile effect via the oxytocin receptor).

Based on the above, it is reasonable to extrapolate that retosiban, through some unknown mechanism, is capable of inhibiting contraction of the myometrium of subjects in which the uterus is overdistended, for example in situations of multiple gestation or polyhydroamnios

The observation that retosiban seems to change the signalling pathways of the myometrium in such a way as to prevent it responding to contractile stimuli may also explain the observation that in a phase II clinical study, retosiban resulted in a greater delay to delivery than other drugs routinely used as tocolytics (see background).

Therefore, in a first aspect, the invention provides a method of treating a human female subject with a multiple gestation or polyhydroamnios in pre-term labour, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject.

In a second aspect, the invention provides a method of treating a human female subject in pre-term labour, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)- 1-methylpropyl]-2,5-piperazinedione to the human female subject, characterized in that the human female subject has a multiple gestation or polyhydroamnios

In a third aspect, the invention provides a method of treating a human female subject in pre-term labour, which method comprises a step of identifying whether said human female subject has a multiple gestation or polyhydroamnios and a step of administering (3R,6R)-3-(2,3-dihydro-1 H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to a human female subject that has a multiple gestation or polyhydroamnios.

In another aspect, the invention provides (3R,6R)-3-(2,3-dihydro-1H-inden-2-yl)-1[(1R)-1-(2-methyl-1,3-oxazol-4-yl)-2-(morpholin-4-yl}-2-oxoethyl]-6-[(1S)-1-methylpropyl]-2,5-piperazinedione for use in the treatment of pre-term labour in a human female subject that has a multiple gestation or polyhydroamnios

In yet another aspect, the invention provide use of (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione in the manufacture of a medicament for the treatment of pre-term labour in a human female subject that has a multiple gestation or polyhydroamnios.

To practice the claimed invention, it is necessary to be able to identify the subject to be treated. In this regard, as discussed in the background section, it is difficult to distinguish labour leading to imminent delivery from “false labour” based on clinical impression alone. In the context of this invention, the subject is considered to be in “labour” if diagnosed as such by a physician or midwife, using diagnostic criteria known in the art. The current criteria are the presence of regular painful uterine contractions in association with dilation and/or effacement of the uterine cervix.

The term pre-term labour relates to spontaneous labour initated before 37 weeks gestational age. Methods of determining gestational age are well known in the art. These include calculation based on the date of the last menstrual period (adjusting for cycle length) and methods based on sonographic measurement of fetal size, such as the crown-rump length of the fetus, or measurement of the head circumference, biparietal diameter or femur length of the fetus, with the exact measurements being used depending on the gestational age when the assessment is made.

In one embodiment, the subject has a multiple gestation. Multiple gestation is where there is more than one fetus in the uterine cavity. Multiple gestation is readily diagnosed by sonography.

In another embodiment, the subject has polyhydroamnios Polyhydroamnios is generally confirmed using ultrasonic measurements. These can be either assessment of the largest measureable vertical pool of amniotic fluid when an ultrasound scan is performed, or measurement of the largest measurable vertical pool of amniotic fluid in each of the four assumed quadrants (upper left and right and lower left and right) of the uterus (the sum of these measurements—in mm—is termed the amniotic fluid index).

Where the largest measurable vertical pool of amniotic fluid is measured, polyhydroamnios can be diagnosed by a measurement that is>8 cm. In a more particular embodiment, polyhydroamnios can be diagnosed by a measurement that is>12 cm. Even more particularly, polyhydroamnios can be diagnosed by a measurement that is>16 cm.

Where the amniotic fluid index is measured, polyhydramnios may be diagnosed when the sum of these measurements (in mm) exceeds a threshold that does not vary with gestational age. This could be>250, >300, >400, or could be based on some other widely used clinical threshold. Alternatively, where the amniotic fluid index is measured, polyhydramnios can be diagnosed when the sum of these measurements (in mm) exceeds a threshold that differs according to gestational age. Several reference ranges exist for the amniotic fluid index at different gestational ages. A suitable reference range for amniotic fluid index at different gestational ages in uncomplicated singleton pregnancies is given in Table 1 (adapted from Hinh and Ladinsky, Int J Gynec Obstet, 2005, 91: 132-136). Polyhydroamnios can be diagnosed by a measurement that is>90^{th} percentile on an appropriate reference range. In a more particular embodiment, polyhydroamnios can be diagnosed by a measurement that is>95^{th} percentile on an appropriate reference range. Even more particularly, polyhydroamnios can be diagnosed by a measurement that is>97.5^{th} percentile on an appropriate reference range. Alternatively, polyhydroamnios may be diagnosed when this measurement exceeds the mean for a given gestational age (based on the clinically accepted estimated date of delivery) by a threshold of multiples of the standard deviation. Thus, in one embodiment, polyhydroamnios can be diagnosed by a measurement that is greater than or equal to the sum of the mean value for the given gestational age (based on the clinically accepted estimated date of delivery) plus 1 standard deviation. In a more particular embodiment, polyhydroamnios can be diagnosed by a measurement that is greater than or equal to the sum of the mean value for the given gestational age (based on the clinically accepted estimated date of delivery) plus 2 standard deviations.

TABLE 1

| Gestational | | Mean | Standard | 90% | 95% | 97.5% |
|---|---|---|---|---|---|---|
| age (wk) | N | (mm) | Deviation | (mm) | (mm) | (mm) |
| 28 | 43 | 139 | 30 | 193 | 199 | 214 |
| 29 | 43 | 151 | 31 | 188 | 195 | 208 |
| 30 | 38 | 132 | 25 | 183 | 190 | 202 |
| 31 | 43 | 135 | 26 | 178 | 185 | 196 |
| 32 | 44 | 129 | 29 | 173 | 180 | 189 |
| 33 | 47 | 135 | 27 | 168 | 175 | 183 |
| 34 | 44 | 137 | 31 | 162 | 170 | 177 |
| 35 | 38 | 126 | 22 | 157 | 166 | 171 |
| 36 | 41 | 111 | 26 | 152 | 161 | 165 |
| 37 | 41 | 121 | 24 | 147 | 156 | 159 |
| 38 | 48 | 114 | 21 | 142 | 151 | 153 |
| 39 | 53 | 118 | 17 | 137 | 146 | 147 |
| 40 | 37 | 110 | 10 | 131 | 141 | 140 |

Polyhydroamnios may also be diagnosed clinically. It could be suspected by an enlarged symphysis-fundal height for gestational age. The suspicion might be confirmed by noting that the uterus appears hard (referred to as tight or tense), when foetal parts are not readily palpated in circumstances (based on gestational age and maternal body habitus) where they might be expected to have been palpable, or through other aspects of physical examination, such as the elicitation of a “fluid thrill”, as described in standard textbooks on the subject.

It is noted that, in the clinical trials conducted to date with retosiban, subjects with multiple gestation and polyhydroamnios were excluded and clinical trials have only been conducted with subjects in spontaneous pre-term labour with uncomplicated singleton pregnancies. The females that are the subject of the methods and uses of the invention are thus distinct from those treated in the retosiban clinical trials. Further, the subjects of the methods and uses of the invention are distinguished from those treated in clinical trials by their physiological status, namely multiple gestation or polyhydroamnios

In certain embodiments of the invention, (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione (henceforth retosiban) is administered parenterally. In a more particular embodiment, retosiban is administered intravenously. Even more particularly, retosiban is administered via IV (intravenous) infusion at a dose required to produce plasma concentrations of retosiban of between 5 and 400 ng/mL. Most particularly, retosiban is administered via IV (intravenous) infusion at a dose required to produce plasma concentrations of retosiban of between 75 and 150 ng/mL.

In certain embodiments where retosiban is administered via IV infusion, a steady state plasma concentration of between 50 to 400 ng is provided for 2 hours. The steady state plasma concentration is then reduced such that this is between 5 to 150 ng at 48 hours from the start of the infusion.

In another embodiment, retosiban may be administered as a 6 mg intravenous loading dose over 5 minutes, followed by a 6 mg/hour continuous infusion over 48 hours. For subjects with an inadequate response after the first hour of treatment, the dose may be increased by another 6 mg loading dose over 5 minutes followed by a 12 mg/hour continuous infusion for the remainder of the 48 hour period.

In other embodiments of the invention, retosiban is administered orally. In a more particular embodiment, retosiban is administered orally at a dose of between 200-500 mg/day. The desired dose may conveniently be presented in a single dose or as divided doses administered at appropriate intervals, for example as two, three, four or more sub-doses per day. In one embodiment, the daily dose is selected to provide plasma concentrations of retosiban of between 5 and 400 ng/mL. More particularly, the daily dose is selected to provide plasma concentrations of retosiban of between 75 and 150 ng/mL.

The experiments shown in the example also show that retosiban reduced the sensitivity to oxytocin (as measured by the pEC₅₀) in both conditions of high and low stretch. This observation suggests that the prophylactic administration of retosiban may be effective in preventing pre-term labour in a subject at risk of pre-term labour.

A subject at risk of pre-term labour is a subject with any recognized risk factor for pre-term labour (certain of these are discussed in the Background section). These include a short cervix (see below for diagnosis), the presence of fetal fibronectin on a vaginal swab, previous pre-term birth, previous cervical surgery, uterine abnormality (diagnosed by sonography), polyhydroamnios and multiple gestation (as discussed above, these last two conditions are associated with uterine overdistension).

A short cervix may be diagnosed by transvaginal sonography or clinical examination. In one embodiment, a short cervix can be diagnosed by a measurement that is≤25 mm In a more particular embodiment, a short cervix can be diagnosed by a measurement that is≤20 mm

Alternatively, a short cervix can be diagnosed by a measured that is less than a threshold (that differs according to gestational age). Several reference ranges exist for cervical length. A suitable reference range for cervical length in singleton pregnancies is given in Table 2 ((adapted from Salomon et al., Ultrasound Obstet Gynecol, 2009, 33: 459-464). A short cervix can be diagnosed by a measurement that is≤10^{th} percentile on an appropriate reference range. In a more particular embodiment, a short cervix can be diagnosed by a measurement that is≤5^{th} percentile on an appropriate reference range. Even more particularly, a short cervix can be diagnosed by a measurement that is≤1^{st} percentile on an appropriate reference range. Alternatively, a short cervix may be diagnosed when this measurement is lower than the mean for a given gestational age (based on the clinically accepted estimated date of delivery) by a threshold of multiples of the standard deviation. Thus, in one embodiment, a short cervix can be diagnosed by a measurement that is less than or equal to the mean value for the given gestational age (based on the clinically accepted estimated date of delivery) minus 1 standard deviation. In a more particular embodiment, a short cervix can be diagnosed by a measurement that is less than or equal to the mean value for the given gestational age (based on the clinically accepted estimated date of delivery) minus 2 standard deviations.

TABLE 2

| Gestational | | Mean | Standard | 1% | 5% | 10% |
|---|---|---|---|---|---|---|
| age (wk) | N | (mm) | Deviation | (mm) | (mm) | (mm) |
| 16 | 79 | 43.3 | 6.7 | 27.0 | 32.2 | 34.9 |
| 17 | 99 | 41.7 | 6.6 | 26.1 | 31.5 | 34.2 |
| 18 | 68 | 42.8 | 6.3 | 25.2 | 30.8 | 33.6 |
| 19 | 61 | 41.5 | 8.2 | 24.2 | 30.1 | 33.0 |
| 20 | 108 | 42.5 | 6.9 | 23.2 | 29.3 | 32.3 |
| 21 | 142 | 41.3 | 8.0 | 22.2 | 28.5 | 31.6 |
| 22 | 979 | 40.3 | 7.5 | 21.1 | 27.7 | 30.9 |
| 23 | 1149 | 40.6 | 7.7 | 19.9 | 26.8 | 30.1 |
| 24 | 309 | 39.7 | 7.8 | 18.6 | 25.8 | 29.2 |
| 25 | 123 | 39.9 | 9.5 | 17.1 | 24.7 | 28.3 |
| 26 | 119 | 36.6 | 9.4 | 15.6 | 23.5 | 27.2 |
| 27 | 136 | 37.2 | 8.3 | 14.2 | 22.4 | 26.2 |
| 28 | 156 | 35.7 | 9.3 | 13.0 | 21.4 | 25.2 |
| 29 | 120 | 35.9 | 9.4 | 11.9 | 20.4 | 24.3 |
| 30 | 79 | 32.8 | 9.1 | 10.9 | 19.5 | 23.5 |
| 31 | 95 | 34.4 | 9.3 | 10.1 | 18.6 | 22.6 |
| 32 | 940 | 34.1 | 8.9 | 9.4 | 17.8 | 21.8 |
| 33 | 1405 | 33.2 | 9.4 | 8.8 | 17.0 | 20.9 |
| 34 | 324 | 30.8 | 9.8 | 8.2 | 16.1 | 19.9 |
| 35 | 65 | 39.3 | 10.4 | 7.7 | 15.2 | 19.0 |
| 36 | 58 | 29.5 | 9.7 | 7.2 | 14.4 | 18.1 |

Therefore, in another aspect, the invention provides a method for preventing pre-term labour in a human female subject with at least one recognized risk factor for pre-term labour which method comprises administering (3R,6R)-3-(2,3-dihydro-1 H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject.

In another aspect, the invention provides a method for preventing pre-term labour in a human female subject, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject, characterized in that the human female subject has at least one recognized risk factor for pre-term labour.

In yet another aspect, the invention provides a method for preventing pre-term labour in a human female subject, which method comprises a step of identifying whether said human female subject has at least one recognized risk factor for pre-term labour and a step of administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to a human female subject that has at least one recognized risk factor for pre-term labour.

In a further aspect, the invention provides a method for preventing pre-term labour in a human female subject with at least one recognized risk factor for pre-term labour selected from a short cervix, the presence of fetal fibronectin on a vaginal swab, previous pre-term birth, previous cervical surgery, uterine abnormality, polyhydroamnios and multiple gestation, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5 -piperazinedione to the human female subject.

In yet another aspect, the invention provides a method for preventing pre-term labour in a human female subject, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject, characterized in that the human female subject has at least one recognized risk factor for pre-term labour selected from a short cervix, the presence of fetal fibronectin on a vaginal swab, previous pre-term birth, previous cervical surgery, uterine abnormality, polyhydroamnios and multiple gestation.

In a further aspect, the invention provides a method for preventing pre-term labour in a human female subject, which method comprises a step of identifying whether said human female subject has at least one recognized risk factor for pre-term labour selected from a short cervix, the presence of fetal fibronectin on a vaginal swab, previous pre-term birth, previous cervical surgery, uterine abnormality, polyhydroamnios and multiple gestation, and a step of administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to a human female subject that has at least one recognized risk factor for pre-term labour selected from a short cervix, the presence of fetal fibronectin on a vaginal swab, previous pre-term birth, previous cervical surgery, uterine abnormality, polyhydroamnios and multiple gestation.

In a further aspect, the invention provides a method for preventing pre-term labour in a human female subject with a multiple gestation or polyhydroamnios, which method comprises administering (3R,6R)-3-(2,3-dihydro-1H-inden-2-yl)- 1-[(1R)-1-(2-methyl-1,3-oxazol-4-yl)-2-(morpholin-4-yl}-2-oxoethyl]-6-[(1S)- 1-methylpropyl]-2,5-piperazinedione to the human female subject.

In yet another aspect, the invention provides a method for preventing pre-term labour in a human female subject, which method comprises administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to the human female subject, characterized in that the human female subject has a multiple gestation or polyhydroamnios

In yet another aspect, the invention provides a method for preventing pre-term labour in a human female subject, which method comprises a step of identifying whether said human female subject has a multiple gestation or polyhydroamnios, and a step of administering (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione to a human female subject that has a multiple gestation or polyhydroamnios

Similarly, the invention also provides (3R,6R)-3-(2,3-dihydro-1H-inden-2-yl)-1-[(1R)-1-(2-methyl- 1,3-oxazol-4-yl)-2-(morpholin-4-yl}-2-oxoethyl]-6-[(1S)-1-methylpropyl]-2,5-piperazinedione for use in the prevention of pre-term labour in a human female subject with at least one recognized risk factor for pre-term labour.

In another aspect, the invention also provides (3R,6R)-3-(2,3-dihydro-1H-inden-2-yl)-1[(1R)-1-(2--methyl-1,3-oxazol-4-yl)-2-(morpholin-4-yl}-2-oxoethyl]-6-[(1S)-1-methylpropyl]-2,5-piperazinedione in the prevention of pre-term labour in a human female subject with at least one recognized risk factor for pre-term labour selected from a short cervix, the presence of fetal fibronectin on a vaginal swab, previous pre-term birth, previous cervical surgery, uterine abnormality, polyhydroamnios and multiple gestation.

In a further aspect, the invention provides (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione in the prevention of pre-term labour in a human female subject with a multiple gestation or polyhydroamnios.

In yet another aspect, the invention provides use of (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione in the manufacture of a medicament for the prevention of pre-term labour in a human female subject with at least one recognized risk factor for pre-term labour.

In another aspect, the invention also provides use of (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione in the manufacture of a medicament for the prevention of pre-term labour in a human female subject with at least one recognized risk factor for pre-term labour selected from a short cervix, the presence of fetal fibronectin on a vaginal swab, previous pre-term birth, previous cervical surgery, uterine abnormality, polyhydroamnios and multiple gestation.

In a further aspect, the invention provides use of (3R,6R)-3-(2,3-dihydro-1H-inden-2-yl)-1-[(1R)-1-(2-methyl-1,3-oxazol-4-yl)-2-(morpholin-4-yl}-2-oxoethyl]-6-[(1S)-1-methylpropyl]-2,5-piperazinedione in the manufacture of a medicament for the prevention of pre-term labour in a human female subject with a multiple gestation or polyhydroamnios

In one embodiment of the methods for preventing pre-term labour, retosiban is administered orally. In a more particular embodiment of these methods, retosiban is administered orally at a dose of between 200-500 mg/day. The desired dose may be conveniently be presented in a single dose or as divided doses administered at appropriate intervals, for example as two, three, four or more sub-doses per day. In one embodiment, the daily dose is selected to provide plasma concentrations of retosiban of between 5 and 400 ng/mL. More particularly, the daily dose is selected to provide plasma concentrations of retosiban of between 75 and 150 ng/mL.

Whilst it is possible that, for use in any of the methods/therapies described above, a compound of the invention may be administered as the raw chemical, it is preferable to present the active ingredient as a pharmaceutical formulation. Formulations suitable for parenteral and oral administration are well known in the art.

### EXAMPLE

Method

Human myometrial strips (obtained from non-labouring patients undergoing routine elective caesarean section at term) were cultured in medium (phenol red free DMEM supplemented with 10% charcoal stripped foetal calf serum, 2 mM L-glutamine and antibiotics) in an incubator (37° C., humidified 5% CO₂) under conditions of low stretch (0.6 g mass) or high stretch (2.4 g mass). Half of the strips in both the low and high stretch groups were incubated with 1 mM retosiban (diluted in DMSO). The remaining strips (paired samples from the same patient) were incubated DMSO.

After 20-24 hours incubation, myometrial contractility of the strips was studied as described previously (J Physiol 2012; 590(Pt9): 2081-2093). Briefly, tension was initially set at 2 g for all strips. Strips were washed with fresh Krebs after 15 and 30 min and the tension reset to 2 g. After a further hour of washes (every 15 min), the issue was treated with 50 mM KCl (50 mM, 5-7 min). This was washed, the tissue allowed to recover and then a cumulative concentration response curve to oxytocin (up to 100 nM) was obtained. Maximal responses to KCl and oxytocin were normalized to strip wet weight. The mean normalized responses of duplicate strips were calculated for the four different groups (low stretch with retosiban, low stretch with vehicle, high stretch with retosiban, high stretch with vehicle). Effects were expressed as fold change i.e. the ratio of normalized responses in the experimental and control conditions from different strips obtained from the same woman. pEC₅₀ values were calculated using analysis of the area under the curve for each concentration of oxytocin.

Since the data were expressed as fold changes, all ratios were log transformed and normal distribution following log transformation was assessed using the Sharpiro-Wilk test. The null hypothesis was that there was no change in the mean normalized responses with a given intervention. All statistical tests were one sample Student's t-tests that the mean fold change was significantly different from one (i.e. all analysis were paired comparisons of different strips from the same woman). Continuous associations were assessed using linear regression of log transformed fold changes. For the analysis of pEC50 Student's paired t-test was used.

Results

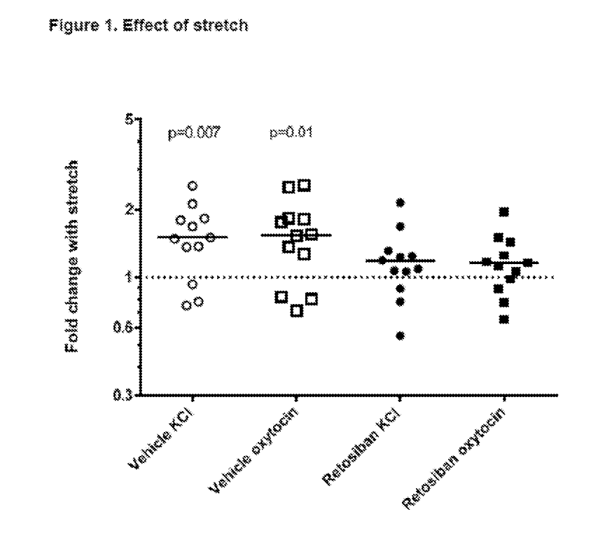

Effect of Stretch on Myometrial Contractility in Tissues Incubated With Retosiban or Vehicle Increased stretch increased the contractility of tissues that were not incubated with retosiban (FIG. 1). The median fold change (IQR, P) with stretch (high stretch compared with low stretch) was 1.59 (1.14-1.81, P=0.007, n=12) and 1.51 (1.04-1.82, P=0.01, n=12) (Student's paired T test after logarithmic transformation) for KCI and oxytocin, respectively. There was no statistically significant effect of stretch when strips were incubated with retosiban (FIG. 1). The mean fold change was 1.14 (0.97-1.27, P=0.27, n=12) and 1.14 (0.94-1.34, P=0.23, n=12) for KCI and oxytocin, respectively.

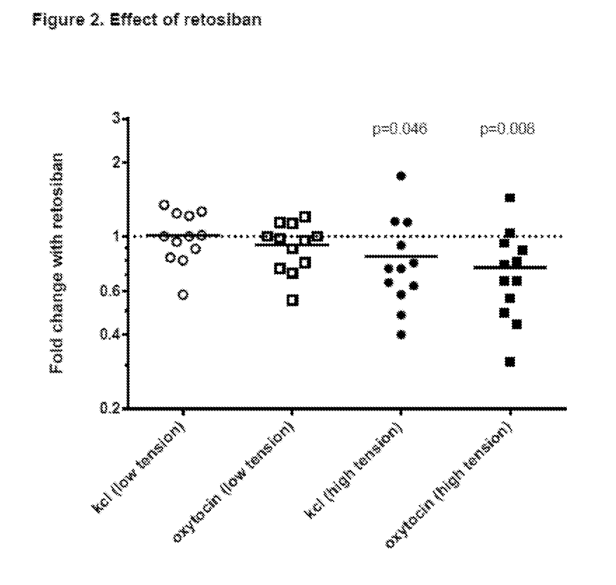

Effect of Incubation with Retosiban in Low and High Stretch In tissues exposed to low stretch, incubation with retosiban had no statistically significant effect on the maximal response to either KCI or oxytocin (FIG. 2). The median fold change (IQR, P) with retosiban was 1.00 (0.85-1.22, P=0.81, n=12) for KCI and 0.97 (0.76-1.07, P=0.15, n=12) for oxytocin. In tissues exposed to high stretch, incubation in retosiban resulted in a statistically significant reduction in the maximal response to both KCI and oxytocin (FIG. 2). The median fold change was 0.74 (0.60-1.03, P=0.046, n=12) for KCI and 0.71 (0.53-0.91, P=0,008, n=12) for oxytocin.

Relationship Between the Effect of Stretch and Effect of Retosiban

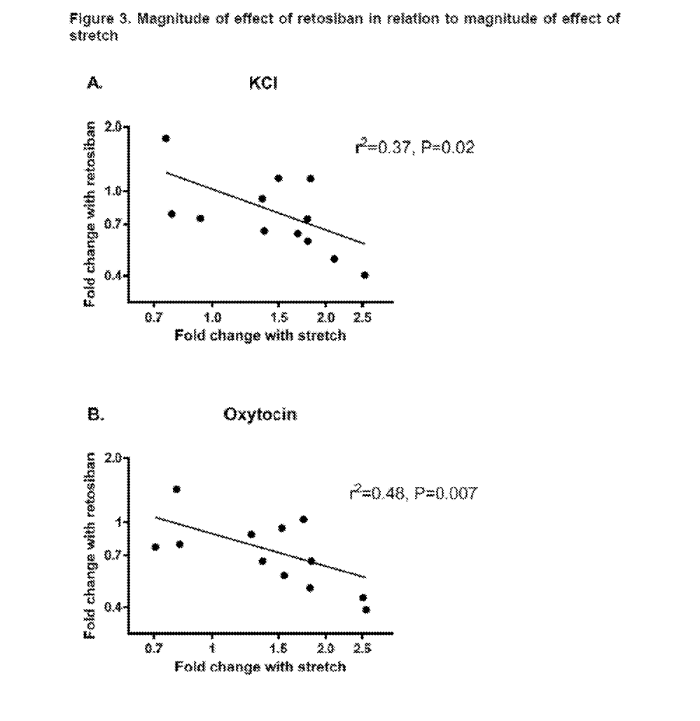

There was significant variation in the magnitude of reduction in responses to KCI and oxytocin induced by incubation in retosiban. The greater the effect of stretch on responses of myometrium from a given patient, the greater was the reduction in response induced by retosiban (FIG. 3).

Effect of Stretch on the pEC₅₀ to Oxytocin

The sensitivity of the myometrium to oxytocin was estimated using the pEC₅₀. Stretch had no significant effect on the pEC50 to oxytocin in the presence of either retosiban or vehicle. In tissues incubated with retosiban, the median pEC₅₀ (IQR) to oxytocin was 8.43 (8.22-8.68) in low stretch and 8.65 (8.34-8.71) in high stretch (P=0.51, n=10). In tissues incubated with vehicle the pEC₅₀ to oxytocin was 8.90 (8.77-9.04) in low stretch and 9.08 (8.86-9.22) in high stretch (P=0.17, n=11).

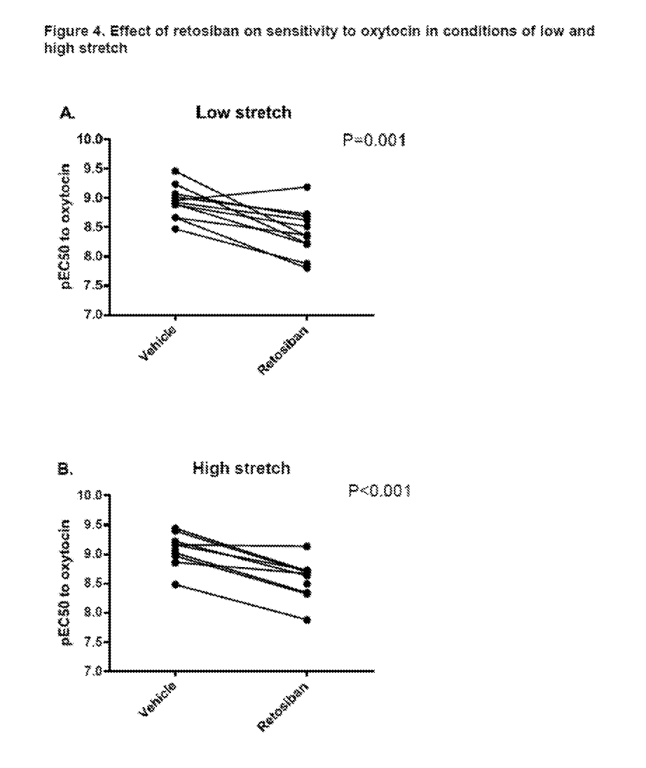

Effect of Retosiban on the pEC₅₀ to Oxytocin in Similarly Stretched Tissues Retosiban reduced the sensitivity to oxytocin in both conditions of low and high stretch (FIG. 4). When incubated under low stretch, the pEC₅₀ to oxytocin in samples incubated in retosiban was 8.36 (8.21-8,68) and in samples incubated in vehicle was 8.93 (8.66-9.07) (P=0.001, n=11). When incubated under high stretch, the pEC₅₀ to oxytocin in samples incubated in retosiban was 8.67 (8.34-8.71) and in samples incubated in vehicle was 9.16 (8.97-9.22) (P<0.001, n=9).

Conclusions

Prolonged stretch of human myometrium increases its contractile response to both potassium and oxytocin.

Incubation in retosiban had no effect on the maximum contractility of myometrium in conditions of low stretch,

Incubation in retosiban inhibited the maximum contractility of myometrium exposed to high stretch and the magnitude of the inhibition increased as the effect of stretch increased

Incubation with retosiban significantly decreased the sensitivity of myometrium to oxytocin under both conditions of low and high stretch.

The effects of retosiban could not simply be explained by oxytocin receptor antagonism as (i) all experiments quantifying contractility were performed in the absence of retosiban, (ii) similar changes were observed in the response to KCI, which stimulates myometrial contractility by direct depolarisation of smooth muscle

These data support the hypothesis that prolonged exposure of myometrial smooth muscle to retosiban may inhibit the pro-contractile effect of stretch

1. A method of treating a human female subject with multiple gestation or polyhydroamnios in pre-term labour wherein the method comprises administering a therapeutically effective amount of (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione to the human female subject.

2. The method according to claim 1 wherein the human female subject has multiple gestation.

3. The method according to claim 1 wherein the human female subject has polyhydroamnios.

4. The method according to claim 1 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered parenterally.

5. The method according to claim 4 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered via intravenous infusion at a dose required to produce plasma concentrations of retosiban of between 5 and 400 ng/mL.

6. The method according to claim 1 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered orally.

7. The method according to claim 6 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered at a dose of between 200-500 mg/day.

8. A method for preventing pre-term labour in a human female subject with at least one recognized risk factor for pre-term labour wherein the method comprises administering a therapeutically effective amount of (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione to the human female subject.

9. The method according to claim 8 wherein the at least one recognized risk factor for pre-term labour is selected from a short cervix, the presence of fetal fibronectin on a vaginal swab, previous pre-term birth, previous cervical surgery, uterine abnormality, polyhydroamnios, and multiple gestation.

10. The method according to claim 9 wherein the at least one recognized risk factor for pre-term labour is polyhydroamnios or multiple gestation.

11. The method according to claim 10 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered orally.

12. The method according to claim 11 wherein (3R,6R)-3-(2,3-dihydro-1H-inden-2-yl)-1-[(1R)-1-(2-methyl-1,3-oxazol-4-yl)-2-(morpholin-4-yl)-2-oxoethyl]-6-[(1S)-1-methylpropyl]-2,5-piperazinedione is administered at a dose of between 200-500 mg/day.
